(19) United States
(12) Patent Application Publication
(10) Pub. No.: US 2011/0245384 A1
(43) Pub. Date: Oct. 6, 2011

(54) Process for the production of a functionalised carbon nanomaterial

(75) Inventors: Alexander Bismarck, London (GB); Milo Sebastian Peter Shaffer, London (GB); Robert Menzel, London (GB); Michael Q. Tran, London (GB); Angelika Menner, London (GB)

(73) Assignee: IMPERIAL INNOVATIONS LIMITED, London (UK)

(21) Appl. No.: 13/002,618

(22) PCT Filed: Jul. 2, 2009
(86) PCT No.: PCT/GB2009/001655

§ 371 Date: Mar. 29, 2011

(30) Foreign Application Priority Data
Jul. 4, 2008 (GB) 0812320.0

(51) Int. Cl. C08K 5/1515; C07D 301/00; C07C 69/54; C07C 69/527; B82Y 30/00

(52) U.S. Cl. 524/114; 549/513; 560/205; 560/183; 977/742

(57) ABSTRACT

The invention provides a process for the production of a functionalised carbon (nano)material comprising heating a carbon (nano)material in an inert atmosphere to produce a surface-activated carbon (nano)material and incubating said surface-activated carbon (nano)material with a chemical species capable of reacting with the surface-activated carbon (nano)material.

The present invention relates to a process for the production of a functionalised carbon (nano)material.

There is currently a vast interest in the art of carbon (nano)materials. This interest stems from the unique property profile of such materials. Carbon nanotubes, for example, possess extremely high mechanical properties (tensile strength and modulus), high thermal conductivity, electrical conductivity ranging from semiconducting to metallic and unique binding properties to biological materials. Carbon (nano)materials, in particular carbon nanotubes, are currently being investigated for use in applications such as nanoreinforcements, gas sensors, thermal emitters, gas sensors, nanoscale electrical devises, catalysts, and coatings.

One of the largest obstacles regarding the use of carbon (nano)materials, and in particular carbon nanotubes and fibres, is the inherent lack of compatibility with many surrounding materials of interest. This lack of interaction and compatibility is mainly due to the graphitic surface structure of the carbon (nano)materials.

In order to overcome this obstacle, functionalisation methods are used to produce sites on carbon (nano)material surfaces that improve the interaction with their environment. The majority of functionalisation methods disclosed in the art involve treatments with aggressive liquid phase reagents, such as mineral acids, carbenes, nitrenes, often followed by a series of further derivatisation reactions. However there are significant disadvantages with these methods. In particular, these methods involve multistep procedures, lengthy purification procedures and generate large amounts of chemical waste. ‘Dry’ approaches include treatment with high reactive fluorine gas or plasma treatments; fluorine is difficult and dangerous to use, whilst plasma treatments treat limited volumes in an uneven fashion with relatively expensive equipment.

An example of a liquid phase method used in the art involves grafting of polymers to the surface of carbon (nano)materials during the polymerisation reaction. However, this method is wasteful as it can generate ungrafted polymer and involves difficult purification steps (for example, separating the polymer grafted material from a solution containing monomer and ungrafted polymer). The procedures disclosed in the prior art involve processes which are costly and time consuming and provide carbon (nano)material which are unsuitable for use in many of the potential applications for these materials.

The present invention provides a method for the functionalisation of carbon (nano)materials which can be used to provide a wide range of surface functional groups. It provides functionalised carbon (nano)materials which can be simply separated and purified. The process can be readily applied to a large volume of material and is broadly compatible with the type of equipment often used to synthesise nanotubes and other carbon nanomaterials

There is therefore provided by the first aspect of the invention, a process for the production of a functionalised carbon (nano)material comprising heating a carbon (nano)material in an inert atmosphere to produce a surface-activated carbon (nano)material and incubating said surface-activated carbon (nano)material with a chemical species capable of reacting with the surface-activated carbon (nano)material.

For the purposes of this invention, the heating of the carbon (nano)material results in the activation of the surface of the carbon (nano)material by the formation of free radicals on the surface of the carbon (nano)material.

Activation of the carbon (nano)material is carried out in an inert atmosphere (i.e. an atmosphere free of oxygen and water) or a vacuum. The inert atmosphere should further be free of any reactive species.

The activation of the carbon (nano)material is carried out at a temperature of 500° C. or above, preferably at a temperature of 800° C. or above. The carbon (nano)materials of the present invention have thermally decomposable functional groups, including for example C—H bonds, particularly oxygen-containing functional groups, such as carbon oxides, on the surface. These thermally decomposable functional groups are either inherently present on the surface of the carbon (nano)material or arise as a result of a number of methods commonly applied to carbon nanomaterials, for example acid oxidation, thermal oxidation, plasma oxidation, etc. It will be appreciated, if used, that the conditions for pre-oxidation of the carbon (nano)material can be selected to allow the formation of surface carbon oxides which decompose to free radicals. Examples of such surface carbon oxides include ketones. In order to activate the carbon (nano)material, the carbon (nano)material should be heated to a temperature at which the thermally decomposable functional groups decompose resulting in the generation of free radicals on the surface of the carbon (nano)material. The activation temperature should therefore exceed the decomposition temperature of the thermally decomposable functional groups present on the surface of the carbon (nano)material (i.e., of those thermally decomposable functional groups which decompose to form free radicals). The minimum activation temperature is therefore determined by the composition of the carbon (nano)material and can be established experimentally, either by assessing the success of the subsequent grafting reaction or using specific analytical methods, for example temperature programmed desorption measurements (TPD) coupled to mass spectroscopy. In general, an activation temperature of 800° C. or above has been determined to be sufficient to allow the required activation. The activation temperature should not however be less than 500° C. The activation temperature can exceed 500° C. (i.e. it can be carried out at 600° C., 700° C., 800° C., 900° C., 1000° C., 1100° C., 1200° C., etc. However, it will be appreciated that the use of temperatures in excess of the preferred temperature of 800° C. will increase the cost of the process of the invention.

The activation temperature can be above, at or below the graphitisation temperature of the carbon (nano)material. The graphitisation temperature will depend on the carbon (nano)material, however graphitisation can commence at a temperature in the region of 1200° C. (although temperatures in the range of 1600 to 2800° C. are more usual). Graphitisation tends to heal surface defects in the carbon (nano)material through reorganization of the carbon lattice and is often considered to improve the quality and intrinsic properties of nano-materials. Therefore, in one aspect of the invention, activation occurs at a temperature at or above the graphitisation temperature of the carbon (nano)material so that activation and graphitisation occur simultaneously. It will be appreciated that graphitisation may reduce the number of sites available for functionalisation on the surface of the carbon (nano)material. Therefore, in an alternative aspect of the invention, the activation is therefore carried out below the graphitisation temperature of the carbon (nano)material to maximise the number of reactive sites.

The removal of the surface functional groups by thermal decomposition leads to the generation of surface free radicals on the carbon surface. This thermal activation takes place in an oxygen and water free inert atmosphere or an ultrahigh vacuum (for example a vacuum of from 10⁻² to 10⁻⁴ mbar) at temperatures at or exceeding 800° C. Vinyl (for example (meth)acrylate) monomers or other reagents capable of reacting with surface free radicals are then brought into contact with the thermally activated carbon material at temperatures around room temperature resulting in functionalisation or polymer grafting away from the carbon surface. The inert atmosphere must be maintained until after the reaction with the monomer has been completed.

For the purposes of this invention, the chemical species is selected from a monomer which is accessible by free radical polymerisation, such as a (meth)acrylate monomer or a vinyl monomer, a polymer, a fluorescent dye, a coupling agent, a surfactant, a free radical tag/trap (such as nitroxides, organic halides and especially organic iodides for example 1-iodododecane) or a free radical initiator (such as azo compounds, persulfates and organic peroxides). The vinyl monomer is preferably one or more selected from the group comprising ethylene, propylene, methyl methacrylate, styrene, (3,5,5-trimethylcyclohex-2-enylidene)malononitrile, 1,1-dichloroethylene, 1-(3-sulfopropyl)-2-vinylpyridinium hydroxide, 1-vinyl-2-pyrrolidinone, vinylnaphthalene 2-isopropenyl-2-oxazoline, 2-vinyl-1,3-dioxolane, vinylnaphthalene, vinylpyridine, 4-vinyl-1-cyclohexene 1,2-epoxide, 4-vinyl-1-cyclohexene, vinylanthracene, vinylcarbazole, divinyl sulfone, ethyl vinyl sulfide, N-ethyl-2-vinylcarbazole, N-methyl-N-vinylacetamide, N-vinylformamide, N-vinylphthalimide, trichlorovinylsilane, vinyl bromide, vinyl chloride, vinylcyclohexane, vinylcyclopentane, vinylphosphonic acid, vinylsulfonic acid, vinyltrimethylsilane, cis-1,3-dichloropropene, vinyl acetate, acrylic acid, acrylonitrile, (dimethylamino) ethylmethacrylate, lauryl methacrylate, 2-(methylthio)ethyl methacrylate, trimethylsilyl methacrylate, 2-hydroxyethyl methacrylate, hydroxy propyl methylacrylate, acrylamide, oleic acid, glycidyl methacrylate (GMA) and maleic anhydride. The resulting functionalised carbon (nano)material can then be readily used in a number of applications where improved dispersion of and/or interaction with the carbon (nano)material is required.

Incubation of the surface-activated carbon (nano)material with the chemical species is preferably carried out at or slightly above room temperature, for example at a temperature of from 10 to 40° C., for example at a temperature of 15 to 35° C., such as 25 to 30° C. It will be appreciated that this temperature range is provided for guidance. The incubation of the surface-activated carbon (nano)material with the chemical and species can be carried out at temperatures below room temperature, provided that the chemical species (which can be in a liquid or gaseous form) do not undergo a phase change to become solid or glassy. Conversely, the upper limit for the incubation of the surface activated carbon (nano)material with the chemical species is the temperature at which the chemical species either decomposes and/or reacts with itself. For example, this upper limit is in the range of 60 to 70° C. for vinyl monomers. It will be appreciated that the use of temperatures near room temperature (for example +/−5° C.) will minimise the cost of the process. In some circumstances, temperatures slightly (i.e. from 1 to 10° C. above room temperature may be selected in order to improve the control of the process.

Purification from residual monomers can be either accomplished by the vacuum-assisted evaporation of the monomer or by conventional filtration and washing. Alternatively or additionally evaporation of the residual monomers can be accelerated by heating at a temperature below the self reaction temperature of the polymer (i.e. to avoid polymerisation of the monomer). The chemical species are preferably gaseous or volatile species. Such gaseous or volatile species allow a simple purification of the functionalised material. The use of a volatile reactive species provides an additional process benefit. The reservoir of liquid volatile reagent can be stabilised by a non-volatile radical scavenger. As the volatile reagent is drawn off as a vapour, it is distilled, leaving the scavenger behind. After passing through the activated carbonaceous material, any unused reagent can be recondensed into the reservoir where it is once again stabilised.

In the case of carbon nanotubes and fibres used for nanocomposites, the choice of either polymerising a monomer, which has a known affinity for the host matrix, or a reactive compatibilising monomer may be used in order to improve interfacial adhesion between carbon nanotubes and fibres, respectively, and the host material. This improvement in adhesion tends to lead to improved mechanical, electrical and thermal performance of the composites containing the functionalised carbonaceous (nano)material.

Specific examples of the first aspect of the invention involve functionalisation of a carbon (nano)material with MMA. Such a functionalised carbon (nano)material could be used as reinforcement in PMMA, polycarbonate or PVDF. Alternatively, a carbon (nano)material, preferably a carbon (nano)tube can be functionalised using HEMA or acrylamide. Such a functionalised carbon (nano)material can be provided for use in polyamides or epoxy systems.

The process of the first aspect of the invention is particularly applicable to carbon nanotubes, carbon fibres, carbon nanotubes and carbon blacks. The functionalised carbon (nano)materials have improved dispersability and compatibility with solvents, polymers and biological media. The claimed process allows for the functionalisation of carbonaceous (nano)materials by grafting a wide range of reactive moieties, for example vinyl monomers to the surface of the carbon (nano)materials without the need for traditional initiators, additional solvents, time consuming purification or separation steps. The surface properties of the carbon can be tailored to meet the compatibility requirements of any host material (i.e. matrices) with applications including, but not limited to, monolithic systems (i.e. carbonaceous (nano)materials used alone), composite systems, biological applications, thermal and electrical devices.

The second aspect of the invention provides a process for the production of a surface activated carbon (nano)material comprising heating a carbon (nano)material in an inert atmosphere such that free radicals are formed on the surface of the carbon (nano)material.

For the purposes of this invention, the activation of the carbon (nano)material is carried out at a temperature of 500° C. or above, preferably 800° C. or above.

The disclosed invention is simple, scalable, can be fully back integrated to existing CVD equipment (commonly used for carbon nanotube growth), and can be employed for sensitive reagents due to the mild reaction conditions. The functionalisation of the carbon (nano)materials is localised on the surface where it is most needed to improve adhesion and interaction with its environment.

The third aspect of the invention provides a functionalised carbon (nano)material as produced by the process of the first aspect of the invention.

The fourth aspect of the invention provides a composite system comprising a functionalised carbon (nano)material described in the third aspect of the invention or as produced by the process of the first aspect of the invention and a matrix. For the purpose of this invention, the matrix can be any material conventionally used in the art to produce composite systems, such as maleic anhydride grafted PVDF. For the purposes of the fourth aspect of the invention, the functionalised carbon (nano)material acts as a reinforcement in the composite system.

All preferred features of each of the aspects of the invention apply to all other aspects mutatis mutandis.

The invention may be put into practice in various ways and a number of specific embodiments will be described by way of example to illustrate the invention with reference to the accompanying drawings, in which:

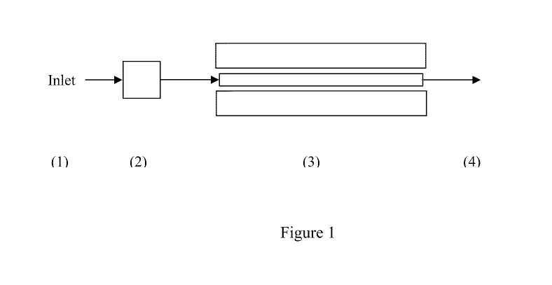

FIG. 1 shows a schematic of the tube furnace setup. (1) N₂ inlet, (2) oxygen scrubber (Cu powder, 400° C.), (3) tube furnace, (4) N₂ outlet and monomer inlet, respectively;

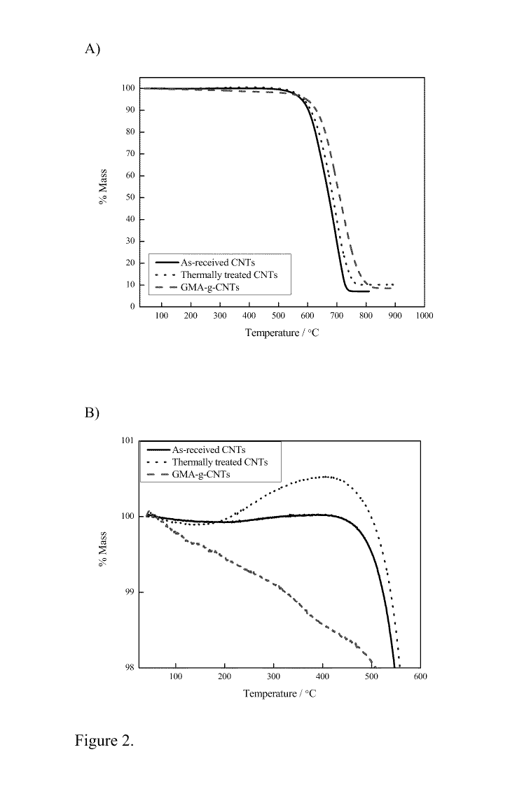

FIG. 2. shows the thermogravimetric analysis of three different nanotubes samples: ‘as received’, thermally treated and exposed to room temperature air, and GMA-grafted. —A) shows the shows the full thermal oxidative profile of the ‘as received’, thermally treated and exposed to room temperature air, and GMA-grafted carbon nanotubes and B) shows the detail of the degradation of the grafted polymer on the GMA-grafted carbon nanotubes, compared to two controls, as discussed in example 1;

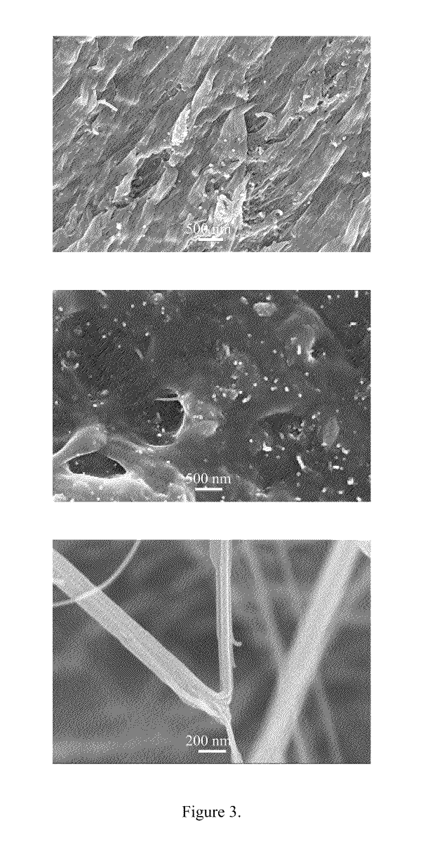

FIG. 3 shows SEM micrographs of the tensile fracture surface of nanotube-PVdF composites based on (3A) ‘as received’ carbon nanotubes and (3B) & (3C) of the GMA-grafted carbon nanotube nanocomposite tensile samples. FIG. 3B) also shows the presence of microfibrils which are present only in the GMA-grafted carbon nanotube nanocomposite. FIG. 3C) shows the presence of GMA-grafted nanotubes within the microfibrils;

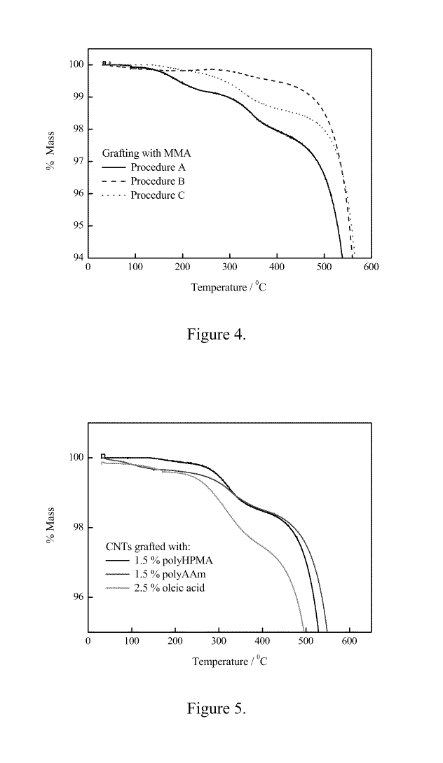

FIG. 4 shows the thermogravimetric analysis of MMA-grafted carbon nanotubes including the grafting content resulting from thermochemical activation (Procedure A), at 30° C. (Procedure B) and at 60° C. (Procedure C);

FIG. 5 shows the thermogravimetric analysis of carbon nanotubes grafted with HPMA, AAm and oleic acid, respectively;

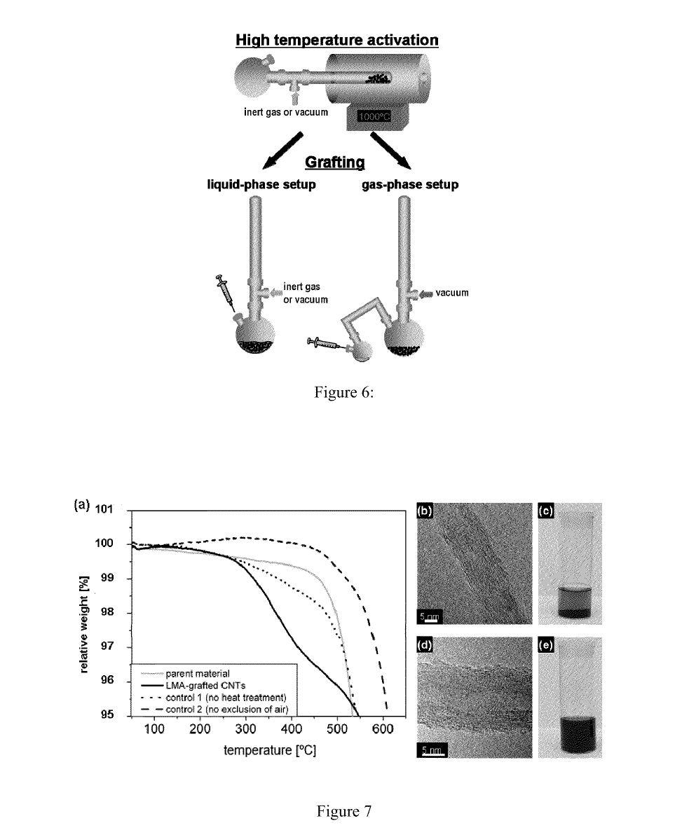

FIG. 6 shows the experimental set up for the thermochemical grafting of nanotubes with functional organic monomers; under inert atmosphere or vacuum, as described in example 3;

FIG. 7 shows the characterisation of CNTs grafted with lauryl methacrylate (LMA): TGA weight loss profiles of LMA-grafted CNTs and corresponding control samples (a); HRTEM images of parent CNT (b) and LMA-grafted CNT (d); dispersion of parent CNTs (e) and LMA-grafted CNTs (e) in butyl acetate;

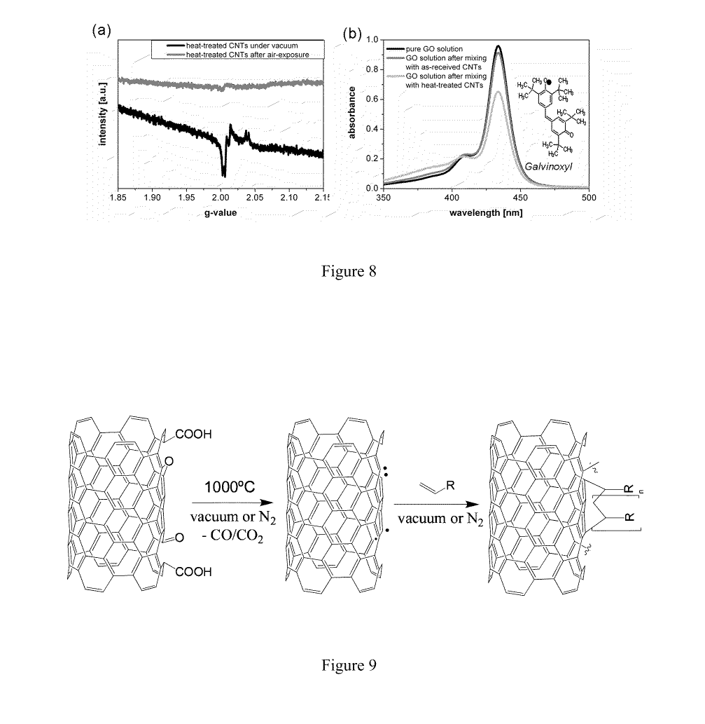

FIG. 8 shows (a) EPR spectra of heat-treated Arkema CNTs in vacuum and after air exposure for 1 h, recorded at 6K; (b) UV-Vis spectra of a pure galvinoxyl (GO) solution in toluene after mixing with heat-activated commercial CNTs and untreated Arkema CNTs under vacuum, respectively;

FIG. 9 shows a proposed mechanism for the thermochemical activation and grafting of CNTs;

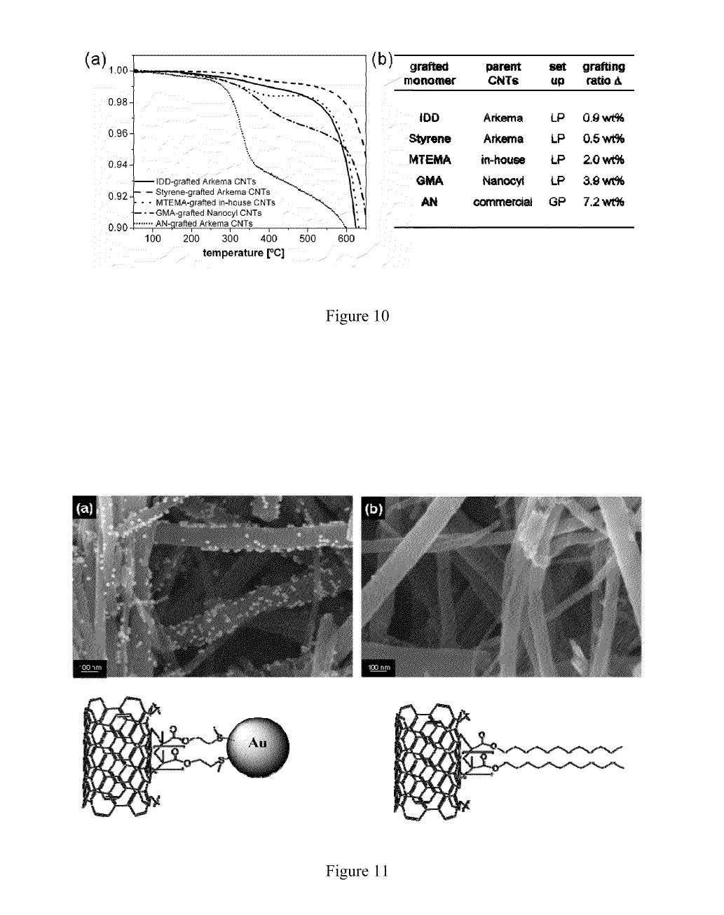

FIG. 10 shows versatility of the thermochemical grafting approach: (a) TGA weight loss profiles, and (b) grafting ratios for commercial and in-house grown CNTs grafted with various organic compounds for (acronyms and structures of the grafted compounds are set out in Table 2);

FIG. 11 shows SEM images of in-house CNTs grafted with (a) MTEMA and (b) LMA after exposure to a dispersion of gold nanoparticles, followed by thorough washing in both cases;

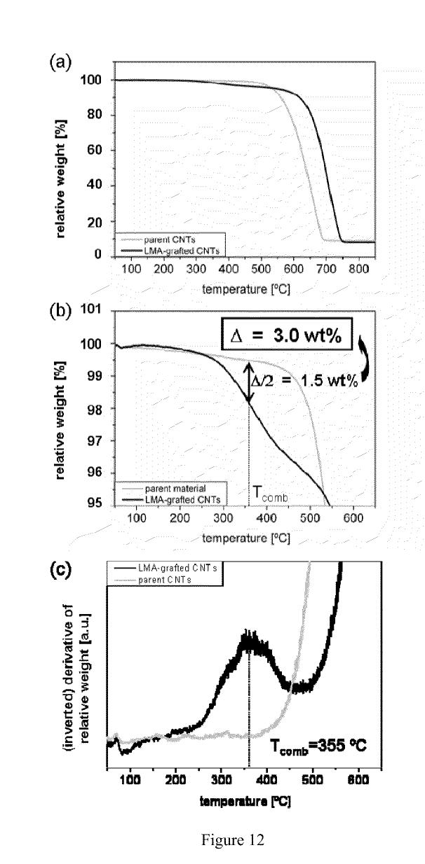

FIG. 12 shows TGA analysis of LMA grafted CNTs illustrating the determination of the combustion temperature of the grafted organic matter, T_{comb}, and the grafting ratio Δ; (a) complete weight loss profiles in the temperature range of 50-850° C.; (b) magnification of (a) in the temperature range of 50-650° C.; (c) derivatives of weight loss profiles in (b);

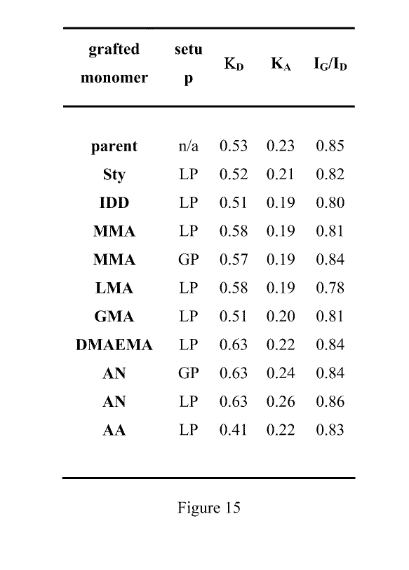

FIG. 15 shows electron acceptor and donor numbers, K_{A} and K_{D}, from IGC measurements, and I_{G}/I_{D} ratio from Raman spectroscopy for commercial CNTs grafted with various functional organic compounds; and

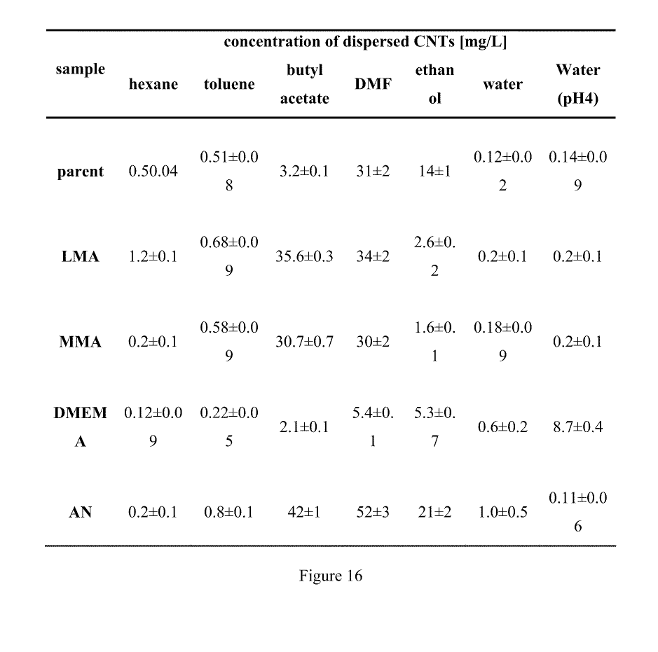

FIG. 16 shows dispersion in different solvents for commercial CNTs grafted with various functional compounds.

### EXAMPLES

### Example 1

### Preparation of Glycidyl Methacrylate (GMA) Grafted Carbon Nanotubes

Directly before use, the inhibitor hydroquinone was removed from commercially available GMA via filtration over a two layered chromatographic column consisting of basic activated and neutral activated alumina. The purified monomer was then purged with argon to remove any dissolved oxygen and water.

Thermally oxidised multi-walled carbon nanotubes were produced by a cutting procedure previously described in Tran, M., Tridech, C., Alfrey, A., Bismarck, A., Shaffer, M., Thermal oxidative cutting of multiwell carbon nanotubes. Carbon 2007, 45, (12) 2341-2350.

The thermal chemical activation of thermally oxidised multi-walled carbon nanotubes as well as the grafting reaction was carried out in a tube furnace in an atmosphere of purified and dry nitrogen (FIG. 1). Nitrogen was passed through a packed bed of copper powder heated to 400° C. (FIG. 1 (2)) to remove any traces of oxygen and water before entering the tube furnace. The nitrogen flow was kept constant at a flow rate of 50 ml/min throughout the duration of the entire experiment. Thermally oxidised multi-walled carbon nanotubes (500 g) were placed into an alumina boat which was placed into the centre of a tube furnace (FIG. 1 (3)) at room temperature and nitrogen was passed over the carbon nanotubes for 1 h. The tube furnace was then heated to 1000° C. for 1.5 h. Afterwards, the entire system was allowed to cool to 30° C. before GMA (5 ml) was injected directly onto the thermally activated carbon nanotubes in nitrogen counter flow (FIG. 1 (4)). The carbon nanotubes/GMA mixture was allowed to react for at least 5 h. The GMA-grafted nanotubes were washed at least three times with acetone and tetrahydrofuran to remove residual monomer. Excess solvent was removed under vacuum.

Since the thermal stability of vinyl polymers is significantly lower than that of the carbon nanotubes, thermogravimetric analysis (TGA) provides a convenient means of determining the quantity of grafted polymer. FIG. 2 shows the thermogravimetric profile of ‘as received’ carbon nanotubes in air, the thermally-treated carbon nanotubes without the addition of monomer and GMA-grafted nanotubes in a temperature range between 20° C. and 900° C. A weight loss of 1.5 wt. % in the range of the decomposition temperature of acrylic polymers (200° C.-400° C.) can be observed for the GMA-grafted nanotubes. In comparison, the ‘as-received’ carbon nanotubes showed less than 0.5 wt. % change, while the weight of thermally treated carbon nanotubes showed a slight mass increase at temperatures between 200° C. and 400° C. The slight increase in mass is likely due to the oxidation of the carbon nanotubes after the oxide desorption procedure, which after exposure to room temperature air is known to produce basic character oxides (see Bismarck, A., Richter, D., Wuertz, C., Springer, J., Basic and acidic surface oxides on carbon fiber and their influence on the expected adhesion to polyamide. Colloids and Surfaces, A, Physicochemical and engineering aspects. 1999, 159, (2), 341.). Additionally, the GMA-grafted nanotubes had a degradation temperature, as determined by the peak rate of mass loss (T_{d}), of 705° C. This value is a significant improvement over the ‘as-received’ carbon nanotubes (T_{d}=670° C.), as well as, the thermally treated carbon nanotubes without the addition of monomer (T_{d}=680° C.). This fact suggests that the thermochemical grafting procedure also provides a more thermally stable termination to the edges of the graphene sheets which constitute the (defective) carbon nanotubes.

### Nanocomposite Preparation:

A maleic anhydride grafted PVDF based nanocomposite containing 2.5 wt.-% GMA-grafted carbon nanotubes (GMA-g-CNT in MAH-g-PVDF) was manufactured. Maleic anhydride grafted PVDF was dissolved in dimethyl formamide (DMF). A suspension of GMA-grafted carbon nanotubes in DMF was prepared by sonication and the appropriate amount was added to the MAH-g-PVDF solution to make 2.5 wt.-% GMA-g-CNT in MAH-g-PVDF. Afterwards, precipitation of nanocomposite particles was induced by the addition of a non-solvent system such as DMF/water (80/20 wt. ratio) or ethanol. The precipitate was filtered and dried under vacuum at 120° C. The nanocomposite powder was hot-pressed into a 0.5 mm thick film. For comparison, a nanocomposite formulation comprising of ‘as-received’ carbon nanotubes in PVDF, as well as PVDF homopolymer were also prepared using the above mentioned procedure. The films were cut into the tensile specimens (ISO 527-2, Type 5B) and the mechanical performance of the nanocomposite was evaluated by tensile testing with a testing speed of 1 mm/min. The fracture surface of the tensile sample was observed by electron microscopy to investigate the effect of GMA grafting on nanocomposite mechanical performance.

### Nanocomposite Mechanical Performance:

The tensile performance of all the nanocomposite formulations are summarised in Table 1.

TABLE 1
Tensile performance

| | Young's Modulus | Tensile Strength |
|---|---|---|
| Sample | (GPa) | (MPa) |
| PVDF | 1.04 | 46.8 |
| As-received carbon nanotubes w/ | 1.20 | 60.7 |
| PVDF | | |
| GMA-g-CNT w/MAH-g-PVDF | 1.40 | 64.7 |

The tensile strength and Young's modulus of the GMA-g-CNT in MAH-g-PVDF increased by 38% and 35%, respectively as compared to the pristine PVDF. This improvement indicates that the GMA-grafted carbon nanotubes successfully reinforce the polymer matrix. Furthermore, the 17% increase in Young's modulus of the GMA-g-CNT/MAH-g-PVDF nanocomposite compared to the ‘as received’ carbon nanotubes/PVDF nanocomposite due to the improved dispersion and interaction between the GMA-grafted carbon nanotubes and the matrix (FIGS. 3A and 3B). The covalent incorporation of the carbon nanotubes into the PVDF matrix via the reaction of the epoxy group of GMA with maleic anhydride (grafted to PVDF) is the likely reason for the improved mechanical performance. The fracture surface of the GMA-g-CNT/MAH-g-PVDF nanocomposite shows the formation of microfibrils which is characteristic of this nanocomposite formulation only (FIG. 3B). Upon close observation of the microfibrils one can see what appears to be carbon nanotubes within the microfibrils (FIG. 3C). This feature clearly suggests that the adhesion is significantly enhanced between the GMA-grafted carbon nanotubes and the matrix.

### Example 2

### Preparation of Methyl Methacrylate Grafted Carbon Nanotubes

The thermal activation of thermally oxidised multi-walled carbon nanotubes as well as the grafting reaction was carried out in a tube furnace in an atmosphere of purified and dry nitrogen (FIG. 1). Nitrogen was passed through a copper powder heated to 400° C. (FIG. 1 (2)) to remove any traces of oxygen and water before entering the tube furnace. The nitrogen flow was kept constant at a flow rate of 50 ml/min during the duration of entire experiment. Thermally oxidised multi-walled carbon nanotubes (500 g) were placed into an alumina boat which was placed into the centre of a tube furnace (FIG. 1 (3)) at room temperature and nitrogen was passed over the carbon nanotubes for 1 h. The tube furnace was then heated to 1000° C. for 1.5 h. Afterwards, the entire system was allowed to cool to 30° C. before freshly purified methyl methacrylate (MMA) (5 ml) was injected directly onto the thermally activated carbon nanotubes in a nitrogen counter flow (Procedure A). The MMA grafted carbon nanotubes were washed at least three times with acetone and tetrahydrofuran to remove residual monomer. Excess solvent was removed under vacuum.

In order to show the efficiency of the grafting procedure, two other samples of thermally oxidised carbon nanotubes were treated with the above mentioned procedure. However, instead of injecting the freshly purified MMA onto the carbon nanotubes, the carbon nanotubes were exposed to air for at least 2 h at room temperature. Afterwards, freshly purified MMA (5 ml) was added to the resulting oxidised carbon nanotubes and the mixture was either kept at 30° C. (Procedure B) or 60° C. (Procedure C). All carbon nanotubes/MMA mixtures were allowed to react for at least 5 h. The modified carbon nanotubes were washed at least three times with acetone and tetrahydrofuran to remove residual monomer. Excess solvent was removed under vacuum.

TGA was used to determine the yield of grafted polymer. FIG. 4 shows the thermal oxidative profiles of the carbon nanotubes modified via Procedures A, B or C in a temperature range between 20° C. and 600° C. A weight loss of 2.3% in the range of the decomposition temperature of acrylic polymers (200° C.-400° C.) can be observed for the carbon nanotubes grafted with MMA via Procedure A. In comparison, the thermally treated carbon nanotubes, which were exposed to MMA at 30° C. after oxidation Procedure B, showed a slight weight loss in this temperature range (less than 0.5 wt.-%). It is fair to assume that any grafting of the carbon nanotubes under the conditions of procedure B is a result of the strong absorption of MMA to the CNT surface or the thermal or photo-initiated polymerisation of MMA. However, the degree of grafting obtained under the conditions of Procedure B is with 0.5 wt.-% low relative to Procedure A. It is therefore fair to assume that neither the absorption of MMA to the CNT surface nor the thermal or photo-initiated polymerisation of MMA significantly contribute to the grafting of carbon nanotubes obtained under the conditions of Procedure A.

The thermally initiated polymerisation of MMA at 60° C. (Procedure C) led to a significantly lower yield of grafting. A weight loss of only 1.4 wt-% can be observed. The results suggest that vinyl monomers such as MMA are effectively grafted from free radicals on the surface of the carbon nanotubes, which are generated through the thermal chemical activation in Procedure A, not via traditional thermal polymerisation.

The TGA thermogram clearly shows the efficiency of thermochemical activation and the disclosed method for grafting vinyl monomers from the carbon nanotube surface. A relatively high fraction of grafted polymer is contained within the sample, in the case of MMA likely due to the relatively high reactivity of the monomer.

### Example 3

### Preparation of CNTS Grafted with Hydroxypropyl Methacrylate, Acrylamide and Oleic Acid, Respectively

The thermal activation of thermally oxidised multi-walled carbon nanotubes as well as the grafting reaction was carried out in a tube furnace in an atmosphere of purified and dry nitrogen (FIG. 1). Nitrogen was passed through a copper powder heated to 400° C. (FIG. 1 (2)) to remove any traces of oxygen and water before entering the tube furnace. The nitrogen flow was kept constant at a flow rate of 50 ml/min during the duration of entire experiment. 500 mg thermally oxidised multi-walled carbon nanotubes were placed into an alumina boat which was placed into the centre of a tube furnace (FIG. 1 (3)) at room temperature and nitrogen was passed over the carbon nanotubes for 1 h. The tube furnace was then heated to 1000° C. for 1.5 h. Afterwards, the entire system was allowed to cool to 30° C. before either 5 mL freshly purified hydroxypropyl methacrylate (HPMA), 2.1 g acrylamide (AAm) dissolved in 5 mL distilled water or 5 mL oleic acid were injected directly onto the thermally activated carbon nanotubes in a nitrogen counter flow (Procedure A). The grafted carbon nanotubes were washed at least three times with acetone or chloroform (oleic acid grafted CNTs) to remove residual reagents. Excess solvent was removed under vacuum.

TGA was used to determine the yield of grafted polymer. FIG. 5 shows the thermal oxidative profiles of the modified carbon nanotubes in a temperature range between 20° C. and 600° C. A weight loss of 1.5% in the range of the decomposition temperature of acrylic polymers (200° C.-400° C.) can be observed for the carbon nanotubes grafted with HPMA and AAm, respectively, while a 2.5% weight can be observed for the carbon nanotubes grafted with oleic acid.

### Example 4

### Preparation of CNTS

CNTs were synthesised employing typical CVD-growth conditions (Andrews et al, Chemical Physics Letters, 1999, 303, 467) yielding mats of relatively straight and aligned, large MWCNTs (outer diameter 80-100 nm, length of a few hundreds micrometres). Commercial, CVD-grown CNTs were obtained from Arkema S A (Lacq-Mourenx, France) and Nanocyl S A (Sambreville, Belgium) and consisted of aggregates of entangled CNTs with outer diameters of around 10-20 nm and lengths at least a few micrometres. Prior to the thermochemical treatment, the CNTs were pre-oxidised by heating in air (640° C., 6×5 min) in order break-up the entangled CNT agglomerates and introduce additional oxygen-containing functional groups onto the CNT surface. These pre-oxidised CNTs are referred to as “parent” CNTs.

TABLE 2
Overview of organic reactants used for CNT grafting.

| | | Boiling | | |
|---|---|---|---|---|
| | | point | Washing | |
| acronym | monomer | [° C.] | agent | Chemical Structure |
| AA | acrylic acid | 139 | water | 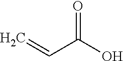 |
| AN | acrylonitrile | 77 | DMF | 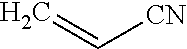 |
| DMAEMA | 2-(dimethyl amino) ethyl methacrylate | 185 | water | 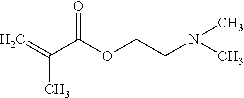 |
| GMA | glycidyl methacrylate | 189 | acetone | 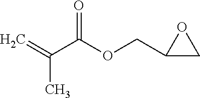 |
| IDD | 1-iodododecane | n/a | toluene | 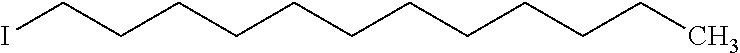 |
| LMA | lauryl methacrylate | 274 | toluene | 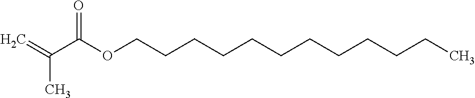 |
| MMA | methyl methacrylate | 101 | toluene | 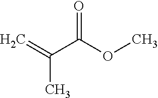 |
| MTEMA | 2-(methylthio) ethyl methacrylate | n/a | THF | 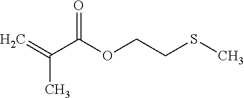 |
| Sty | styrene | 145 | toluene | 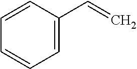 |

The grafting was carried out in a custom-made setup consisting of a 30 mm diameter quartz tube attached to a sample flask (FIG. 6). In order to work under air-free conditions, the setup was connected to an inert gas source or a vacuum system. 100 mg CNTs were heated to 1000° C. under oxygen-free nitrogen or vacuum (5·10⁻⁴ mbar) at 15 K/min in a conventional three-zone tube furnace (PTF 12/38/500, Lenton Ltd, UK) and held at that temperature for 2 h. In a second step, the quartz tube was slowly removed from the heating zone and allowed to cool to room temperature. The CNTs were transferred to the round bottom flask by gravity. 3 mL of the reactant were injected either directly into the flask containing the thermally-activated sample (liquid-phase setup) or into an empty reservoir attached to the sample flask (gas-phase setup). After leaving the sample-monomer mixture under inert atmosphere or vacuum for 12 h, non-reacted reactant was removed via filtration. Prior to characterisation, the product was thoroughly washed three times in order to remove any physisorbed reactants. Each washing step consisted of bath sonication for 5 min in 50 mL of the washing agent (listed for the various reactants in Table 2), filtration and rinsing with 3×50 mL. It should be noted that the washing steps were required for the detailed fundamental study set out below; for application, simple removal and recovery of excess reactant via evaporation under vacuum, without using any solvents, would be sufficient.

Thermogravimetric analysis (TGA) was carried out using a Perkin-Elmer Pyris 1 TGA. Experiments were performed on (2±0.1) mg of CNT material under air flow (flow rate 10 mL/min) applying a constant ramping rate of 10 K/min in a temperature range between 50 and 850° C. The grafting ratio Δ, i.e. the weight of the chemisorbed organic monomer relative to the total weight of the sample, was determined from the height of the first step-like feature in the TGA weight loss profile of the grafted CNTs. The surface coverage of the CNTs, Θ, was estimated from the ratio of the surface area of the CNTs, A_{CNT}, and the surface area of a monolayer of the grafted reactant molecules, A_{grafted}:

$$Θ = \frac{{A}_{grafted}}{{A}_{CNT}} = \frac{{N}_{A} \cdot a \cdot {n}_{grafted}}{m \cdot {S}_{BET}} \qquad ( 1 )$$

where N_{A} is Avogadro's number, m the weight of the CNTs, and S_{BET} the specific surface area of the CNTs as determined by BET measurements. The molar amount of monomer grafted to the surface, n_{grafted} was calculated from the grafting ratio Δ. The cross-sectional area of the organic reactant, a_{react}, was estimated from the density, ρ_{react}, and its molecular weight, M_{react}, using the following equation:

$${a}_{reactant} = \frac{2}{3} \sqrt{\frac{{ρ}_{react}}{{M}_{react}}} \qquad ( 2 )$$

SEM images were obtained on a GEMINI LEO 1525 FEGSEM at an accelerating voltage of 5 kV; TEM images were obtained on a JEOL 2000FX, operating at 200 kV. Samples were dispersed in methanol, and deposited onto an alumina stub or a holey carbon film, respectively. Raman spectra of CNT powders were collected in a range between 1000 and 2200 cm⁻¹ on a LabRam Infinity Raman spectrometer, using a 532 nm laser. The I_{G}/I_{D} ratio was determined from the ratio of surface areas under the Raman bands at around 1580 cm⁻¹ (G-band) and at around 1350 cm⁻¹ (D-band). A large I_{G}/I_{D} ratio indicates pronounced graphitic crystallinity and implies a small defect concentration.

For the dispersion experiments, 5 mg CNTs were sonicated in 5 mL solvent for 30 min and then centrifuged at 10000 rpm for 15 min in order to sediment non-dispersed CNTs. The absorbance of the supernatant was measured on a Lambda 950 spectrometer (Perkin, UK) at 800 nm, and the CNT concentration was determined using Lambert-Beer's Law employing an extinction coefficient of 35.10 mg mL⁻¹ cm⁻¹. Inverse gas chromatography (IGC) allows the determination of the dispersive surface energy (γ^{d}), reflecting CNT surface properties purely due to London forces, and the calculation of acceptor and donor numbers (K_{A} and K_{D}), quantifying the ability of the CNT surface to undergo electron accepting and donating interactions. IGC measurements were carried out in a gas chromatograph (Surface Measurement Systems Ltd., UK), at constant conditions which were chosen to obtain reliable and comparable results. The CNT samples were preconditioned in the IGC column at 120° C. for 2 h before each measurement to ensure that surface contaminants were driven off. IGC tests employed a column temperature of 100° C., a flow rate of 10 mL/min and an injection volume of 1.125 mL. All measurements were performed using helium as carrier gas and methane as inert reference (both gases purchased from BOC, UK). Adsorbate vapours were generated from the organic liquid (HPLC grade, purchased from Sigma-Aldrich); n-hexane, n-heptane and n-octane were used for the quantification of the dispersive surface energy, and ethanol, butanone, ethylacetate and 1,4-dioxane were employed for the determination of the acceptor and donor numbers. IGC results presented are average values of three measurements. For Böhm's titration, 2.5 mL of 0.05 M aqueous sodium hydroxide solution were added to 50 mg CNTs in a polypropylene vial. The CNT suspension was sonicated for 2 hours and further mechanically agitated on an orbital shaker for 4 days. The mixture was then filtered through a polypropylene membrane filter (0.2 mm pore size) and back titrated against 0.01 M aqueous hydrochloric acid solution under nitrogen to restrict any CO₂ absorption.

X-band (9 GHz) continuos-wave Electron paramagnetic resonance (cw-EPR) spectra were recorded with a Bruker ESP300 spectrometer equipped with a high sensitivity resonator (SHQEWO401). Temperatures were adjusted between room temperature and 4 K by a helium cryostat (Oxford ESR 910). Conditions used were as follows: Microwave frequency 9.39 GHz; microwave power, 20 mW; modulation frequency, 100 kHz; modulation amplitude, 0.2 mT.

For the radical quenching experiment, 4.8 mg of galvinoxyl were dissolved in 100 mL dry toluene, and 10 mL of the solution were added to 10 mg of thermally-activated and untreated commercial CNTs, respectively, under vacuum in the liquid-phase experimental setup, and left to react for 1 h. 200 μl of reacted solution were diluted by 2 mL of dry toluene and the UV-Vis spectra were recorded on a Perkin Elmer 950 UV-Vis spectrometer between 350 and 550 nm. Tagging of the grafted in-house CNTs with gold particles was carried out by sonicating around 0.5 mg CNTs in 2 mL methanol for 10 min, followed by addition of 1 mL of aqueous dispersion of 20 nm gold colloids (used as purchased from Sigma-Aldrich) and further sonication for 10 min. A few drops of the resulting dispersion were deposited on an aluminium stub. After drying in air overnight, the CNTs deposit was repeatedly rinsed with water to remove excess gold particles.

Commercial CNTs were high-temperature activated and treated with lauryl methacrylate (LMA) in oxygen-free nitrogen using the liquid-phase experimental setup (FIG. 6) followed by washing with toluene. Thermogravimetric analysis (TGA) of the LMA-treated sample in air confirmed successful grafting (FIG. 7(a)).

Prior to the combustion of the CNTs at around 600° C., a small but distinct weight loss was observed at around 355° C. (FIG. 7(a)), associated with a clear peak in the derivative curve and indicating the combustion of the grafted organics. Two control experiments were carried out under identical treatment conditions. For control experiment 1, the parent CNTs were mixed with the LMA monomer under inert gas omitting the high-temperature treatment; for control experiment 2, the heat-treated CNTs, which had been exposed to a flux of air for 1 h, were mixed with LMA. The products underwent the same washing procedure as applied for the LMA-grafted CNTs. The first control showed a very small, broad weight-loss, with no peak in the derivative; the feature can be attributed to modest physisorption on the heterogeneous CNT surface, caused either by the adsorption of LMA monomer in slit pores or on iron impurities inherently present in these CNTs. In the second control, the slightly rising profile and increased thermal stability of the CNTs is consistent with the presence of basic surface oxides, as observed previously on similar materials. The consistently different weight loss profiles (FIG. 7(a)), therefore, confirm that high-temperature activation and the exclusion of air are prerequisites for successful LMA grafting. From the TGA weight loss profile (FIG. 7(a)), the LMA grafting ratio (i.e. the weight of the chemisorbed organic monomer relative to the total weight of the product) can be estimated to be 3.0 wt % which roughly equates to a CNT surface coverage of around 20% (eq. 1).

FIG. 12 shows TGA analysis of LMA grafted CNTs illustrating the determination of the combustion temperature of the grafted organic matter. T_{comb} and the grafting ratio Δ. In the first derivative of the TGA weight loss profile, the two peaks corresponding to the combustion of the grafted organic matter and the CNTs, respectively, were usually not entirely separated, indicating that the oxidation of the grafted oligomers was not completed at the onset of the CNT combustion. Therefore, the grafting ratio Δ could not directly be determined from the height of the corresponding TGA step feature, but was estimated as double of the weight loss at the combustion temperature, T_{comb}, of the grafted organic matter (FIG. 12(b)). T_{comb} was determined at the peak maximum of the corresponding peak in the first derivative of the TGA trace (FIG. 12(c)).

Note that LMA-grafting improves the combustion resistance of the CNTs significantly (see shift of the complete TGA trace to higher temperatures in FIG. 12(a), due to protection of pre-existing defect sites.

After three repeats, the reproducibility of LMA-grafting reaction was estimated to be Δ=(2.8±0.8) wt %. Sample characterisation by IGC revealed a clear change in CNT surface character after grafting. After LMA-grafting, the dispersive surface energy, γ^{d}, of the CNTs is significantly reduced from (113±2) mJ/m² to (87±2) mJ/m², which is consistent with the occupation or replacement of high-energy sites on the CNT surface with organic monomers. In addition, the K_{D}/K_{A} ratio increased from 2.3±0.1 to 3.1±0.1, indicating a more pronounced electron-donating surface character due the introduction of methacrylic units onto the CNT surface. These changes in dispersive and specific surface character are sufficient to markedly alter dispersion behaviour. Compared to the parent material, the dispersibility of the LMA-grafted CNTs in butyl acetate increased by a factor of ten, from 3 to 35 mg/L (FIG. 7(c) and (e)), but substantially decreased by a factor of five in ethanol (FIG. 11(b)).

The control experiments (FIG. 7(a)) showed that reactive sites are generated during the heat activation step (unlike control experiment 1) but are quenched when exposed to air (control experiment 2). The nature of these reactive sites was further studied using EPR spectroscopy, which allows the detection of species with unpaired electrons. The EPR spectrum of the heat-treated commercial CNTs in vacuum was featureless at room temperature (Supporting Information), but exhibited a relatively narrow signal (g-factor of around 2.01) at a measurement temperature of 6 K. This temperature dependence of the signal intensity is indicative of exchange interactions between conduction electrons and localised spins, such as radicals and paramagnetic ions. The EPR signal is quenched when the CNTs were exposed to air. These observations support the hypothesis that radicals form on the CNT surface, associated with the desorption of surface oxides at high temperatures.

Further quenching studies were carried out to estimate the radical concentration using galvinoxyl, an air-stable radical with a characteristic UV-Vis absorption band at 434 nm. The absorption intensity of galvinoxyl in toluene only marginally changed when mixed with as-received CNTs but significantly decreased when added to the heat-activated CNTs (FIG. 8(b)) presumably due to the binding of galvinoxyl radicals from solution to the radicals on the CNT surface. By assuming that one galvinoxyl radical is quenched by one surface radical, the concentration of the active sites on the CNT can be calculated to 31 μmol per gram CNTs. In a second, independent, quenching experiment, thermally-activated CNTs were reacted with iodododecane resulting in the grafting of 0.9 wt % of organic matter (FIG. 10). Again assuming a stoichiometric reaction, this grafting ratio corresponds to a radical concentration of 50 μmol/g. The two independent quenching experiments indicate similar surface radical concentrations; an average value of 40 μmol/g will therefore be used as an estimate for the concentration of grafting sites on the CNT surface. The grafting site concentration is significantly lower than the surface concentration of oxygen-containing groups on the parent CNTs which has been determined to be about 150 μmol/g by Böhm's titration with NaOH. This difference suggests that only certain types of surface oxides, or other groups, are precursors for the radicals while the main fraction of the functional groups desorb in a heterolytic fashion or undergo migration and restructuring processes during the high temperature treatment. At a grafting ratio of 2.8 wt % LMA, i.e. 110 μmol LMA per gram CNT, there are about three times more monomeric units than grafting sites present on the CNT surface, implying that radical polymerisation of the methacrylate has been initiated by the radicals at the Arkema CNT surface (“grafting from” mechanism). Termination of the polymerisation process might occur either through trace impurities in the reaction system, or via recombination of the propagating chain with a second radical site, resulting in oligomer loops on the CNT surface (FIG. 9). The latter option is likely to be favoured kinetically. On this basis, the covalently-bound LMA oligomers can be estimated to consist of six monomer repeats.

The proposed grafting mechanism implies that the generation of the reactive sites on the CNT surface does not cause any significant additional damage to the graphitic network beyond the original oxidation; this assumption is confirmed by Raman measurements, which yield similar I_{G}/I_{D} ratios for the parent (0.85±0.7) and LMA-grafted (0.81±0.6) materials.

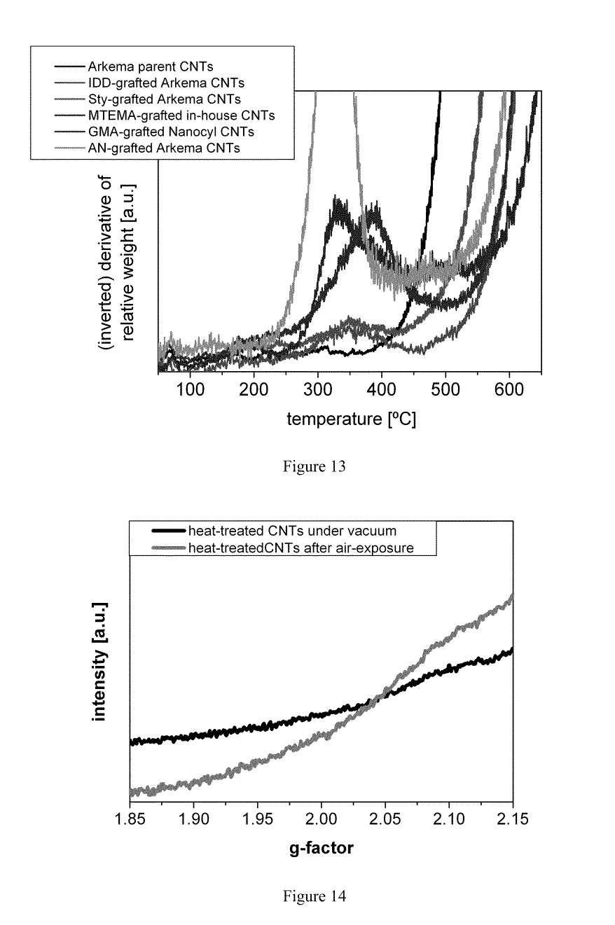

The underlying radical mechanism of the grafting reaction suggests that the thermochemical treatment approach is a generic methodology for the surface modification of CNTs. The generality was, therefore, tested using CNTs of different dimensions and morphologies, and various reactants capable of reacting with radicals, including methacrylates, styrenes, and organic iodides (FIG. 10 and Table 3). Derivatives of the TGA profiles in FIG. 10 are illustrated in FIG. 13.

A tagging reaction was used to determine how the reactive sites are distributed along the CNTs. In-house produced MWCNTs were grafted with both LMA and 2-(methylthio) ethyl methacrylate (MTEMA). By tagging the sulphur groups in MTEMA with gold colloids, the markedly different surface character of the two modified samples was confirmed (FIG. 11). While SEM images show binding of the gold particles to the MTEMA-grafted CNTs, no tagging of the LMA-grafted control sample is observed. The location of the gold colloids in FIG. 6 visualizes the distribution of the grafting sites on the CNT surface. Grafting occurs along the whole length of the nanotubes and is probably associated with the presence of graphene edges and defects sites in the CNT sidewalls.

Various other functional vinyl compounds were grafted onto commercial CNTs. The grafting ratios and average oligomer chain lengths significantly varied with the monomer used and reached values up to 8 wt % and around 70 monomeric units, respectively, for the most reactive compounds (Table 3).

TABLE 3
Overview of commercial CNTs grafted with various functional organic
compounds and some of their properties, including average chain
length of the grafted oligomers (assuming formation of oligomer loops),
dispersive surface energy (standard deviations σ < 2 mJ/m²), ratio of
electron donor and acceptor numbers K_{D}/K_{A} (σ < 0.2), and
dispersibility data (σ < 2 mg/L).

| | | | | disp. | | concentration of | | |
|---|---|---|---|---|---|---|---|---|
| | | | | surface | | dispersed CNTs | | |
| | | grafting | Mono- | energy | | [mg/L] | | |
| grafted | set | ratio Δ | meric | γ^{d} | K_{D}/ | butyl | | water |
| compound | up | [wt %] | units | [mJ/m²] | K_{A} | acetate | ethanol | (pH4) |
| Parent | n/a | n/a | n/a | 113 | 2.3 | 3.2 | 14.0 | 0.1 |
| CNTs | | | | | | | | |
| Sty | LP | 0.5 | 2 | 108 | 2.5 | | | |
| IDD | LP | 0.9 | 1 | 101 | 2.7 | | | |
| MMA | GP | 2.3 | 10 | 83 | 3.0 | | | |
| MMA | LP | 2.5 | 12 | 81 | 3.2 | 30.7 | 1.6 | 0.2 |
| LMA | LP | 2.8 | 6 | 87 | 3.1 | 35.6 | 2.6 | 0.2 |
| GMA | LP | 3.0 | 6 | 84 | 2.6 | | | |
| DMAEMA | LP | 5.2 | 16 | 85 | 2.9 | 2.1 | 5.3 | 8.7 |
| AN | GP | 7.2 | 68 | 81 | 2.6 | 42 | 21 | 0.1 |
| AN | LP | 7.3 | 68 | 77 | 2.5 | | | |
| AA | LP | 7.9 | 54 | 60 | 1.9 | | | |

For acronyms and structures of grafted compounds see Table 2.

Depending on the functionality of the grafted compound, CNT dispersibility was improved in various solvents across a broad spectrum of solvent polarity (Table 3). For instance, the introduction of methyl metacrylate (MMA) oligomers lead to significantly increased dispersibility in butylacetate but reduced dispersion in the more polar ethanol. On the other hand, grafting of the CNTs with 2-(dimethylamino) ethyl methacrylate (DMAEMA) resulted in poor dispersion in butyl acetate but markedly improved dispersibility in acidic aqueous solution due the electrostatic stabilisation of CNTs by protonated amine groups. Grafting of different functional monomers gives rise to altered dispersive and specific surface characteristics of the CNTs, as measured by IGC (Table 3). The dispersive surface energy roughly correlates with the grafting ratio; with increasing coverage of the highly energetic graphitic surface, γ^{d} decreased. Changes in the K_{D}/K_{A} ratios after grafting indicated altered abilities to undergo specific interactions due to the introduction of new functional surface groups. For instance, relative to the parent material, the K_{D}/K_{A} ratio decreased for CNTs modified with acrylic acid (AA), indicating a more electron accepting character, but increased for the DMAEMA-grafted CNTs, implying a more electron donating surface.

The thermochemical modification treatment of the present invention provides a number of technological advantages over conventional wet-chemical CNT functionalisation strategies. It is a versatile and solvent-free “one-pot” reaction approach which is easily scalable. The treatment can be carried out without creating any chemical waste; depending on the application, excess monomer may either remain in the final product or be removed though evaporation under vacuum making time-consuming filtration and washing procedures redundant. The grafting efficiency in the liquid setup was determined to be at least 99% for the MMA-grafted commercial CNTs, i.e. less than 1% of the original monomer was lost due to formation of homopolymer. The high grafting efficiency can be attributed to initiation and propagation of the grafting reaction through surface-bound radical intermediates. CNTs can also be modified in the gas-phase when comparatively volatile monomers, such as MMA and acrylonitrile (AN), are used under vacuum conditions. This particular setup has the advantage that the inhibitor does not have to be removed from the monomer reservoir. Consequently, un-reacted monomer remains stabilised against self-polymerisation and can be reused directly. The gas-phase reaction approach can potentially be extended to reactants with lower vapour pressures when the whole reaction system is kept at elevated temperatures. The grafting ratios, as determined by TGA, and surface properties, as determined by gas chromatography, are comparable to the corresponding products obtained using the liquid-phase setup (Table 3).

1. A process for the production of a functionalised carbon (nano)material comprising heating a carbon (nano)material in an inert atmosphere or vacuum to produce a surface-activated carbon (nano)material and incubating said surface-activated carbon (nano)material with a chemical species capable of reacting with the surface-activated carbon (nano)material.

2. The process as claimed in claim 1 wherein the carbon (nano)material is thermally activated by the formation of free radicals on the surface of the carbon (nano)material.

3. The process as claimed in claim 1 wherein the activation of the carbon (nano)material is carried out at a temperature of 500° C. or above.

4. The process as claimed in claim 1 wherein the activation of the carbon (nano)material is carried out at a temperature of 800° C. or above.

5. The process as claimed in claim 1 wherein the chemical species is selected from a vinyl monomer, a (meth)acrylate monomer, a polymer, a fluorescent dye, a coupling agent, a surfactant, a free radical tag/trap or a free radical initiator.

6. The process as claimed in claim 5 wherein the chemical species is one or more selected from the group comprising ethylene, propylene, methyl methacrylate, styrene, vinyl acetate, acrylic acid, 2-hydroxyethyl methacrylate, glycidyl methacrylate (GMA) maleic anhydride, hydroxypropyl, methacrylate, acrylamide, oleic acid, acrylonitrile, (dimethylamino)ethyl methacrylate, 1-iodododecane, lauryl methacrylate, and 2-(methylthio)ethyl methacrylate.

7. The process as claimed in claim 1 wherein the incubation of the surface-activated carbon (nano)material with the chemical species is carried out at room temperature.

8. The process as claimed in claim 1 wherein the chemical species is a volatile species or is in the gaseous phase.

9. The process as claimed in claim 1 wherein any unreacted chemical species is removed by the application of vacuum and/or heating below the self-reaction temperature of the chemical species.

10. A process for the production of a surface activated carbon (nano)material comprising heating a carbon (nano)material in an inert atmosphere such that free radicals are formed on the surface of the carbon (nano)material.

11. The process as claimed in claim 10 wherein the activation of the carbon (nano)material is carried out at a temperature of 500° C. or above.

12. A functionalised carbon (nano)material as produced by the process of claim 1.

13-16. (canceled)

17. A composite system comprising a functionalised carbon (nano)material as produced by the process of claim 1 and a matrix.
